(19) United States
(12) Patent Application Publication
(10) Pub. No.: US 2024/0148928 A1
(43) Pub. Date: May 9, 2024

(54) SCENT DISPENSING DEVICE AND SCENT REMOVING METHOD

(71) Applicant: Sony Group Corporation, Tokyo (JP)

(72) Inventors: Shuji Fujita, Tokyo (JP); Yukito Inoue, Tokyo (JP); Kazutaka Takaki, Kanagawa (JP)

(73) Assignee: Sony Group Corporation, Tokyo (JP)

(21) Appl. No.: 18/283,072

(22) PCT Filed: Feb. 18, 2022
(86) PCT No.: PCT/JP2022/006604

§ 371 Date: Sep. 20, 2023

(30) Foreign Application Priority Data
Mar. 30, 2021 (JP) 2021-057196

(51) Int. Cl. A61L 9/12; A45D 34/02; A45D 44/00

(52) U.S. Cl. CPC A61L 9/122; A45D 34/02; A45D 44/00; A61L 2209/111; A61L 2209/134

(57) ABSTRACT

To provide a scent dispensing device capable of appropriately and quickly removing an unnecessary scent component remaining in the device after dispensing a scent.

A scent dispensing device 100 includes: a scenting portion 101 that emits a scent-containing air flow containing a scent; a space portion 102 that causes the scent-containing air flow emitted from the scenting portion 101 to pass or remain; a releasing portion 103 that releases the scent-containing air flow having passed through the space portion 102 to a user M; an exhaust portion 104 that exhausts the scent-containing air flow remaining in the space portion 102 to the outside; and a scent component trap portion 105 that traps a volatile component in the scent-containing air flow to be exhausted by the exhaust portion 104.

### TECHNICAL FIELD

The present technology relates to a scent dispensing device and a scent removing method, and more particularly to a scent dispensing device that dispenses a scent to a user by an air flow containing a scent and a scent removing method for removing a remaining scent after dispensing the scent.

### BACKGROUND ART

Conventionally, there has been proposed a scent dispensing device including both a means for releasing a gas containing a scented substance in the vicinity of a user to dispense a scent to the user and a deodorizing means for preventing remaining of an odorous substance.

For example, Patent Document 1 proposes an aroma device that includes an aroma head capable of releasing a gas containing a scented substance at a nose tip of a subject and performing exhaust for deodorization. Furthermore, Patent Document 1 proposes that, in the aroma device described above, a pipe for supplying an odorless gas used to release the gas containing the scented substance and a pipe for intaking air to perform deodorization are integrated with a free arm that holds an aroma head at a position in the air.

### CITATION LIST

### Patent Document

Patent Document 1: Japanese Patent Application Laid-Open No. 2005-304608

### SUMMARY OF THE INVENTION

### Problems to be Solved by the Invention

In the technology of Patent Document 1, however, the means for performing deodorization is only the exhaust, and thus, it is difficult to sufficiently remove a residual scent released into the device or the periphery thereof so that there is a possibility of causing contamination of the scent or an obstacle to accurate olfactory detection of the scent. Furthermore, there is a possibility of causing contamination or damage to an aroma diffuser or peripheral devices due to an aromatic component.

Therefore, a main object of the present technology is to dispense a scent dispensing device capable of appropriately and quickly removing an unnecessary scent component remaining in the device after dispensing a scent. Here, the “scent” includes all kinds of odors such as a good scent and a bad scent.

### Solutions to Problems

The present technology provides a scent dispensing device including: a scenting portion that emits a scent-containing air flow containing a scent; a space portion that causes the scent-containing air flow emitted from the scenting portion to pass or remain; a releasing portion that releases the scent-containing air flow having passed through the space portion to a user; an exhaust portion that exhausts the scent-containing air flow remaining in the space portion to the outside; and a scent component trap portion that traps a volatile component in the scent-containing air flow exhausted by the exhaust portion.

Furthermore, the present technology provides a scent removing method including: a step of emitting a scent-containing air flow containing a scent; a step of causing the emitted scent-containing air flow to pass through an enclosed space; a step of exhausting the scent-containing air flow remaining in the enclosed space to the outside; and a step of trapping a volatile component in the scent-containing air flow to be exhausted.

### Effects of the Invention

According to the scent dispensing device of the present technology, an unnecessary scent component remaining in the device after dispensing the scent can be appropriately and quickly removed. Note that effects described above are not necessarily limited, and any of the effects described in the present specification or other effects that can be grasped from the present specification may be exhibited in addition to or in place of the effects described above.

### BRIEF DESCRIPTION OF DRAWINGS

FIG. 1 is a schematic configuration diagram illustrating a configuration example of a scent dispensing device according to a first embodiment of the present technology.

FIG. 2 is a schematic configuration diagram illustrating an operation example of the scent dispensing device according to the first embodiment of the present technology.

FIG. 3 is a schematic configuration diagram illustrating a configuration example of a scent dispensing device according to a second embodiment of the present technology.

FIG. 4 is a flowchart illustrating an operation example of the scent dispensing device according to the second embodiment of the present technology.

FIG. 5 is a schematic configuration diagram illustrating a configuration example of a scent dispensing device according to a third embodiment of the present technology.

FIG. 6 is a schematic configuration diagram illustrating a configuration example of a scent dispensing device according to a fourth embodiment of the present technology.

FIG. 7 is a schematic configuration diagram illustrating a configuration example of a scent dispensing device according to a fifth embodiment of the present technology.

FIG. 8 is a schematic configuration diagram illustrating a configuration example of a scent dispensing device according to a sixth embodiment of the present technology.

FIG. 9 is a schematic configuration diagram illustrating a configuration example of a scent dispensing device according to a seventh embodiment of the present technology.

FIG. 10 is a schematic plan view illustrating a configuration example of a deodorizing filter according to the seventh embodiment of the present technology.

FIG. 11 is a schematic configuration diagram illustrating an operation example of the scent dispensing device according to the seventh embodiment of the present technology.

FIG. 12 is a schematic configuration diagram illustrating an operation example of the scent dispensing device according to the seventh embodiment of the present technology.

FIG. 13 is a schematic configuration diagram illustrating an operation example of the scent dispensing device according to the seventh embodiment of the present technology.

FIG. 14 is a schematic configuration diagram illustrating a configuration example of a scent dispensing device according to an eighth embodiment of the present technology.

FIG. 15 is a schematic configuration diagram illustrating a configuration example of a deodorizing fan according to the eighth embodiment of the present technology.

FIG. 16 is a schematic configuration diagram illustrating a configuration example of a scent dispensing device according to a ninth embodiment of the present technology.

FIG. 17 is a schematic configuration diagram illustrating a configuration example of a scent dispensing device according to a tenth embodiment of the present technology.

FIG. 18 is a schematic configuration diagram illustrating a configuration example of a scent dispensing device according to an eleventh embodiment of the present technology.

FIG. 19 is a schematic plan view illustrating an arrangement example of an intake portion and an exhaust portion according to the eleventh embodiment of the present technology.

FIG. 20 is a flowchart illustrating an operation example of the scent dispensing device according to the eleventh embodiment of the present technology.

FIG. 21 is a schematic configuration diagram illustrating a configuration example of the scent dispensing device according to the eleventh embodiment of the present technology.

FIG. 22 is a schematic configuration diagram illustrating an operation example of the scent dispensing device according to the eleventh embodiment of the present technology.

### MODE FOR CARRYING OUT THE INVENTION

Hereinafter, preferred modes for carrying out the present technology will be described with reference to the drawings. Embodiments to be described hereinafter illustrate examples of representative embodiments of the present technology, and any embodiments can be combined. Furthermore, the scope of the present technology is not narrowly construed based on these. Note that description will be given in the following order.

1. First Embodiment

(1) Configuration Example of Scent Dispensing Device

(2) Operation Example of Scent Dispensing Device

2. Second Embodiment

(1) Configuration Example of Scent Dispensing Device

(2) Operation Example of Scent Dispensing Device

3. Third Embodiment

4. Fourth Embodiment

5. Fifth Embodiment

6. Sixth Embodiment

7. Seventh Embodiment

(1) Configuration Example of Scent Dispensing Device

(2) Configuration Example of Deodorizing Filter

(3) Operation Example of Scent Dispensing Device

8. Eighth Embodiment

(1) Configuration Example of Scent Dispensing Device

(2) Configuration Example of Deodorizing Fan

(3) Operation Example of Scent Dispensing Device

9. Ninth Embodiment

10. Tenth Embodiment

11. Eleventh Embodiment

(1) Configuration Example of Scent Dispensing Device

(2) Arrangement Examples of Intake Portion and Exhaust Portion

(3) Operation Example of Scent Dispensing Device

12. Twelfth Embodiment

(1) Configuration Example of Scent Dispensing Device

(2) Operation Example of Scent Dispensing Device

### 1. First Embodiment

(1) Configuration Example of Scent Dispensing Device

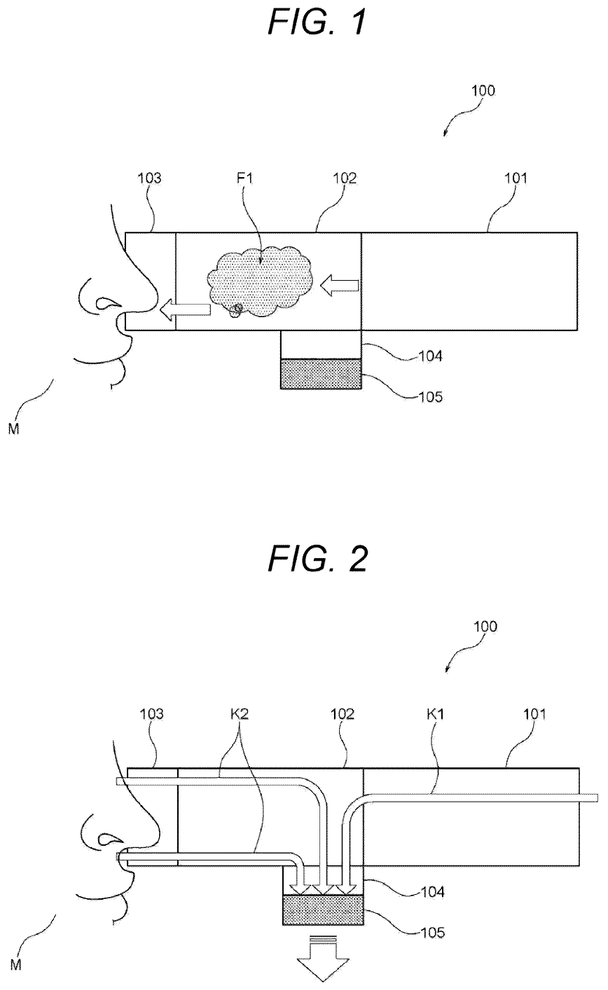

A configuration example of a scent dispensing device 100 according to a first embodiment of the present technology will be described with reference to FIG. 1. FIG. 1 is a schematic configuration diagram illustrating the configuration example of the scent dispensing device 100 according to the present embodiment.

As illustrated in FIG. 1, the scent dispensing device 100 applicable to an olfactory display or the like includes, as an example, a scenting portion 101, a space portion 102 connected to the scenting portion 101, a releasing portion 103 connected to the space portion 102, an exhaust portion 104 attached to the space portion 102, and a scent component trap portion 105 attached in an exhaust direction of the exhaust portion 104.

The scenting portion 101 includes, for example, a perfume retaining member (perfume cartridge) that retains a perfume, which is a substance serving as a source of a scent to be dispended to a user M, and emits a scent-containing air flow containing the scent.

The space portion 102 is an enclosed space that causes the scent-containing air flow emitted from the scenting portion 101 to pass or remain. The space portion 102 may have a guide portion that guides the scent-containing air flow toward the scenting portion 101 and/or a nose tip of the user M. Furthermore, the space portion 102 can also include a mixing mechanism and a blending mechanism of a plurality of types or a plurality of directions of scent-containing air flows.

The releasing portion 103 releases the scent-containing air flow having passed through the space portion 102 to the user M. The releasing portion 103 may have, for example, a detachable nose cover that covers the nose tip of the user M. The nose cover includes, for example, disposable paper, wood, coal, plastic, or the like.

For example, the exhaust portion 104 is attached to a lower portion of the space portion 102 and includes an exhaust fan therein. The exhaust portion 104 exhausts residual scent components F1 in the scent-containing air flow remaining in the space portion 102 to the outside of the scent dispensing device 100 in conjunction with the operation of discharging the scent-containing air flow.

The scent component trap portion 105 is attached to, for example, a lower portion of the exhaust portion 104 in the exhaust direction, and traps a scent component that is a volatile component in the scent-containing air flow exhausted by the exhaust portion 104. The scent component trap portion 105 is, for example, a deodorant or an adsorbent, and may be one in which a gas adsorbing substance such as activated carbon is woven or kneaded or a fiber or a structure body itself has a gas adsorption structure, or may be a filter structure through which an air flow always passes until being discharged. Note that the scent component trap portion 105 may be attached to an upper portion of the exhaust portion 104.

As a material of the scent component trap portion 105, activated carbon (palm, fir, bamboo, resin, wood, fiber, coal, etc.), zeolite, a layered compound (sheet silicate, alumina), porous silica (including mesoporous silica), and the like can be used. Furthermore, a shape of the scent component trap portion 105 may be powdery, granular, fibrous, molded, sheet-like (nonwoven fabric or woven fabric), bar-shaped, plate-shaped, or the like. Furthermore, regarding a surface treatment of the scent component trap portion 105, it does not matter whether or not the activation treatment is performed, and it does not matter whether or not a surface functional group exposure treatment (acidification, alkalization, polarization, or non-polarization) is performed.

(2) Operation Example of Scent Dispensing Device

Next, an operation example of a deodorization process by the scent dispensing device 100 will be described with reference to FIG. 2. FIG. 2 is a schematic configuration diagram illustrating the operation example of the scent dispensing device 100. Note that the procedure of the operation described hereinafter can be performed in a changed order.

First, a scenting operation of dispensing a scent to the user M by the scent dispensing device 100 will be described. First, a perfume retainer is made to retain a perfume of the scent to be dispensed to the user M and a perfume cartridge having the perfume retainer is incorporated into the scenting portion 101.

Thereafter, air flow emission is turned on to cause an air flow to flow into the scenting portion 101. The air flow flowing into the scenting portion 101 flows to the vicinity of the perfume cartridge and comes into contact with the perfume, whereby a scent-containing air flow K1 containing the scent is formed.

The scent-containing air flow K1 flows into the space portion 102, passes through the space portion 102, which is the enclosed space, flows into the releasing portion 103, and is emitted from the releasing portion 103 to the user M. Then, the air flow emission is turned off to end the release to the user M, and at the same time, an operation of the deodorization process of the residual scent components F1 is started.

In the operation of the deodorization process, as an example, in a first step, the exhaust fan of the exhaust portion 104 is turned on simultaneously when the air flow emission is turned off. Note that the exhaust fan of the exhaust portion 104 may be turned on after the air flow emission is turned off.

In a second step, as illustrated in FIG. 2, when the exhaust fan is turned on, the scent-containing air flow K1 remaining on the scenting portion 101 side and a scent-containing air flow K2 remaining on the releasing portion 103 side pass through the space portion 102 and are sucked into the exhaust portion 104 attached to the lower portion of the space portion 102. At this time, the residual scent components F1 remaining in the space portion 102 are also sucked into the exhaust portion 104.

In a third step, the scent-containing air flow K1 and the scent-containing air flow K2, which have been sucked into the exhaust portion 104 and contain the residual scent components F1, are sent into the scent component trap portion 105 such that a scent component is trapped by the scent component trap portion 105 and deodorized, and then, are released to the outside.

As described above, according to the scent dispensing device 100 of the present embodiment, the operation of discharging the scent-containing air flow K1 and the scent-containing air flow K2 containing the residual scent components F1 is started at the same time as the end of the scent dispensing operation, and these air flows always pass through the scent component trap portion 105 until being discharged to the outside, and thus, an unnecessary scent component remaining in the scent dispensing device 100 after dispensing the scent can be appropriately and quickly removed.

Note that the scent dispensing device 100 according to the present embodiment can have a function as a deodorizer by sucking an air flow around the user as well as dispense the scent. Moreover, the scent dispensing device 100 can prevent droplets of saliva of the user from flowing into and adhering to the scent dispensing device 100 by an air shutter by the air flow.

### 2. Second Embodiment

(1) Configuration Example of Scent Dispensing Device

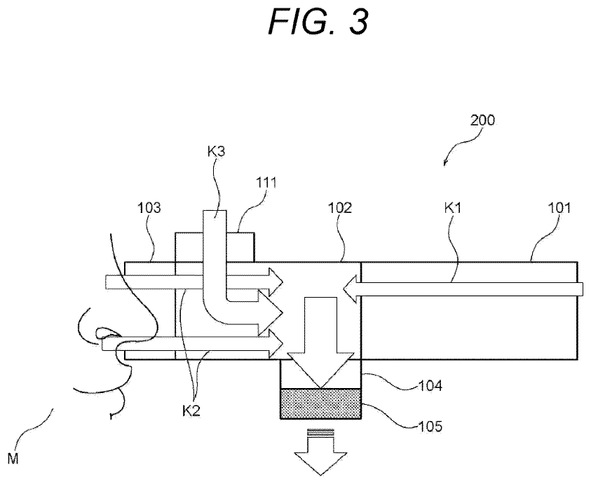

Next, a configuration example of a scent dispensing device 200 according to a second embodiment of the present technology will be described with reference to FIG. 3. FIG. 3 is a schematic configuration diagram illustrating the configuration example of the scent dispensing device 200. The scent dispensing device 200 differs from the scent dispensing device 100 according to the first embodiment in terms of including an intake portion that sucks an external air flow.

As illustrated in FIG. 3, the scent dispensing device 200 includes, as an example, the scenting portion 101, the space portion 102 connected to the scenting portion 101, the releasing portion 103 connected to the space portion 102, the exhaust portion 104 attached to the space portion 102, and the scent component trap portion 105 attached to a lower portion of the exhaust portion 104 in an exhaust direction. Moreover, the scent dispensing device 200 further includes an intake portion 111 attached to the space portion 102.

The intake portion 111 is attached to, for example, an upper portion of the space portion 102 on a side close to the releasing portion 103, and includes an intake fan therein. The intake portion 111 intakes an external air flow K3 into the space portion 102 in conjunction with the exhaust portion 104.

(2) Operation Example of Scent Dispensing Device

Next, an operation example of a deodorization process by the scent dispensing device 100 will be described with reference to FIG. 3.

In the operation of the deodorization process of the scent dispensing device 200, in a first step, an exhaust fan of the exhaust portion 104 and the intake fan of the intake portion 111 are turned on simultaneously when a scenting operation is ended and an air flow emission is turned off.

In a second step, when the exhaust fan and the intake fan are turned on, the scent-containing air flow K1 remaining on the scenting portion 101 side and the scent-containing air flow K2 remaining on the releasing portion 103 side pass through the space portion 102 and are sucked into the exhaust portion 104 attached to the lower portion of the space portion 102. At this time, the residual scent components F1 remaining in the space portion 102 are also sucked into the exhaust portion 104.

In a third step, the external air flow K3 taken by the intake portion 111 also passes through the space portion 102 and is sucked into the exhaust portion 104 attached to the lower portion of the space portion 102 in a state of containing the residual scent components F1 remaining in the space portion 102.

In a fourth step, the scent-containing air flow K1, the scent-containing air flow K2, and the external air flow K3, which have been sucked into the exhaust portion 104 and contain the residual scent components F1, are sent into the scent component trap portion 105 such that a scent component is trapped by the scent component trap portion 105 and deodorized, and then, are released to the outside.

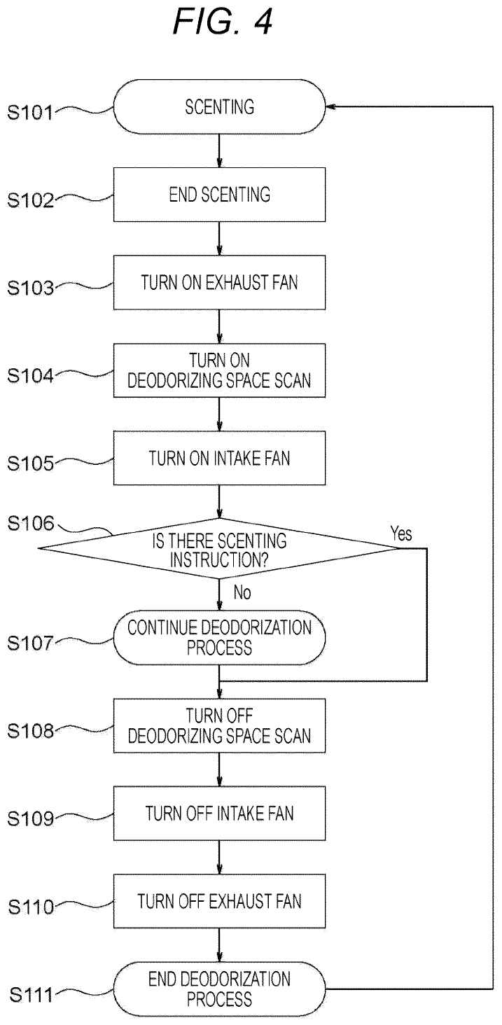

Next, an operation example of the deodorization process in a case where a plurality of the scent dispensing devices 100 is used in conjunction with each other will be described with reference to FIG. 4. FIG. 4 is a flowchart illustrating an operation example of the plurality of scent dispensing devices 100.

As illustrated in FIG. 4, in step S101, a scent-containing air flow formed in the scenting portion 101 is caused to pass through the space portion 102 and be released from the releasing portion 103 to dispense a scent to the user M (scenting).

In step S102, the air flow emission is turned off to stop the scenting operation, performed by releasing of the scent-containing air flow, and the dispensing of the scent to the user M is ended.

In step S103, the exhaust fan of the exhaust portion 104 is turned on in conjunction with the end of the scenting operation.

In step S104, the deodorizing filter 141 turns on deodorizing scan of the space portion 102 and performs the deodorization process in the space portion 102.

In step S105, the intake fan of the intake portion 111 is turned on in conjunction with the turning-on of the exhaust fan of the exhaust portion 104, and the external air flow K3 is taken into the space portion 102 from the intake portion 111.

In step S106, it is determined whether or not there is a scenting instruction from the scent dispensing device 100 different from the scent dispensing device 100 that is performing the deodorization process. In the case of Yes, the flow proceeds to step S108. In the case of No, the flow proceeds to step S107 to continue the deodorization process.

In step S108, the deodorizing scan of the space portion 102 is turned off after the deodorization process in the space portion 102 has been sufficiently performed.

In step S109, the intake fan of the intake portion 111 is turned off.

In step S110, the exhaust fan of the exhaust portion 104 is turned off.

In step S111, the deodorization process is ended. After the end of the deodorization process, the flow returns to step S101 again to start the deodorization process.

According to the scent dispensing device 200 of the present embodiment, the external air flow K3 is also sucked and deodorized so that more unnecessary scent components can be appropriately removed, in addition to effects similar to those of the scent dispensing device 100 according to the first embodiment.

### 3. Third Embodiment

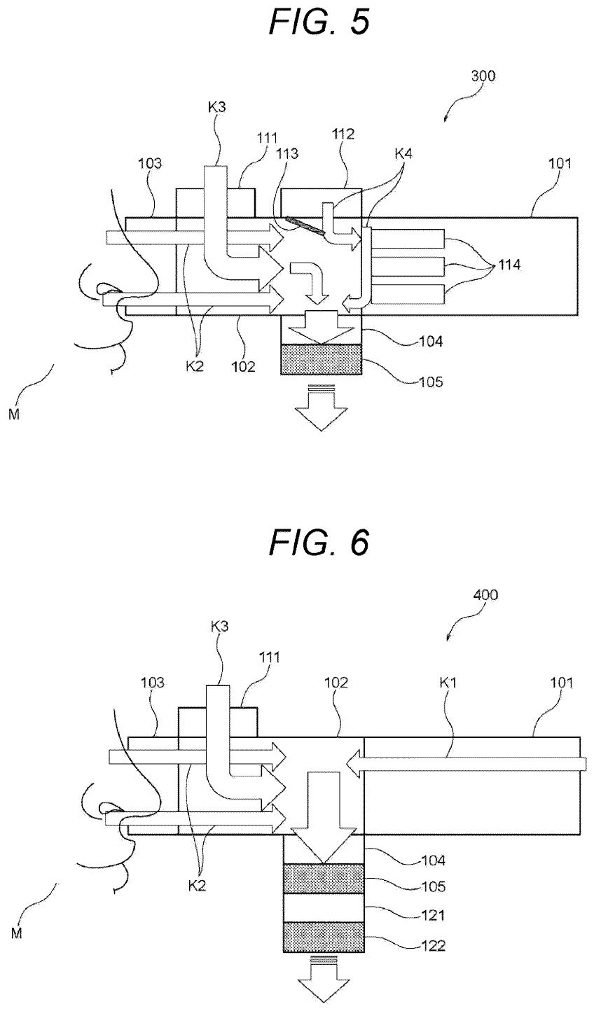

Next, a configuration example of a scent dispensing device 300 according to a third embodiment of the present technology will be described with reference to FIG. 5. FIG. 5 is a schematic configuration diagram illustrating the configuration example of the scent dispensing device 300. The scent dispensing device 300 differs from the scent dispensing device 200 according to the second embodiment in terms of including a plurality of intake portions sucking external air flows.

As illustrated in FIG. 5, the scent dispensing device 300 includes, as an example, the scenting portion 101, the space portion 102 connected to the scenting portion 101, the releasing portion 103 connected to the space portion 102, the exhaust portion 104 attached to the space portion 102, and the scent component trap portion 105 attached to a lower portion of the exhaust portion 104 in an exhaust direction. The scenting portion 101 includes one or a plurality of perfume cartridges 114 each retaining a perfume.

Moreover, the scent dispensing device 200 further includes the intake portion 111 and an intake portion 112 attached to the space portion 102. The intake portion 111 is attached to, for example, an upper portion of the space portion 102 on a side close to the releasing portion 103, and includes an intake fan therein. The intake portion 111 intakes an external air flow K3 into the space portion 102 in conjunction with the exhaust portion 104.

The intake portion 112 is attached to, for example, an upper portion of the space portion 102 on a side close to the scenting portion 101, and includes an intake fan therein. The intake portion 112 takes an external air flow K4 into the space portion 102 in conjunction with the intake portion 111 and the exhaust portion 104. Moreover, the intake portion 112 includes, in the vicinity of an inlet thereof, a flap 113 which is a rectifying mechanism that blows the taken external air flow K4 toward an outlet of the perfume cartridge 114 emitting a scent-containing air flow of the scenting portion 101. The flap 113 brings the external air flow K4 taken from the intake portion 112 into contact with the outlet of the perfume cartridge 114 to remove residual scent components adhering thereto.

In the operation of the deodorization process of the scent dispensing device 300, in a first step, an exhaust fan of the exhaust portion 104 and the intake fan of the intake portion 111 are turned on simultaneously when a scenting operation is ended and an air flow emission is turned off.

In a second step, when the exhaust fan and the intake fan are turned on, the scent-containing air flow K1 remaining on the scenting portion 101 side and the scent-containing air flow K2 remaining on the releasing portion 103 side pass through the space portion 102 and are sucked into the exhaust portion 104 attached to the lower portion of the space portion 102. At this time, the residual scent components F1 remaining in the space portion 102 are also sucked into the exhaust portion 104.

In a third step, the external air flow K3 taken by the intake portion 111 also passes through the space portion 102 and is sucked into the exhaust portion 104 attached to the lower portion of the space portion 102 in a state of containing the residual scent components F1 remaining in the space portion 102.

In a fourth step, the external air flow K4 taken by the intake portion 112 is caused to pass through the outlet of the perfume cartridge 114 by the flap 113, and is sucked into the exhaust portion 104 attached to the lower portion of the space portion 102 in a state of containing the residual scent components F1 adhering to the outlet.

In a fifth step, the scent-containing air flow K1, the scent-containing air flow K2, the external air flow K3, and the external air flow K4, which have been sucked into the exhaust portion 104 and contain the residual scent components F1, are sent into the scent component trap portion 105 such that a scent component is trapped by the scent component trap portion 105 and deodorized, and then, are released to the outside.

According to the scent dispensing device 300 of the present embodiment, the residual scent component at the outlet of the perfume cartridge 114 is also sucked into the external air flow K4 and deodorized so that more unnecessary scent components can be more appropriately removed, in addition to effects similar to those of the scent dispensing device 200 according to the second embodiment.

### 4. Fourth Embodiment

Next, a configuration example of a scent dispensing device 400 according to a fourth embodiment of the present technology will be described with reference to FIG. 6. FIG. 6 is a schematic configuration diagram illustrating the configuration example of the scent dispensing device 400. The scent dispensing device 400 differs from the scent dispensing device 200 according to the second embodiment in terms of including a plurality of exhaust portions and a plurality of scent component trap portions.

As illustrated in FIG. 6, the scent dispensing device 400 includes, as an example, the scenting portion 101, the space portion 102 connected to the scenting portion 101, the releasing portion 103 connected to the space portion 102, the exhaust portion 104 attached to the space portion 102, and the scent component trap portion 105 attached to a lower portion of the exhaust portion 104 in an exhaust direction. Furthermore, the scent dispensing device 400 also includes the intake portion 111 attached to an upper portion of the space portion 102 on a side close to the releasing portion 103.

Moreover, the scent dispensing device 400 further includes an exhaust portion 121 attached to a lower portion of the scent component trap portion 105 in the exhaust direction, and a scent component trap portion 122 attached to a lower portion of the exhaust portion 121 in the exhaust direction. The exhaust portion 121 and the scent component trap portion 122 have functions similar to those of the exhaust portion 104 and the scent component trap portion 105, respectively. Note that any number of exhaust portions and any number of scent component trap portions may be attached depending on applications without being limited to the present embodiment.

In the operation of the deodorization process of the scent dispensing device 400, in a first step, an exhaust fan of the exhaust portion 104 and an intake fan of the intake portion 111 are turned on simultaneously when a scenting operation is ended and an air flow emission is turned off.

In a second step, when the exhaust fan and the intake fan are turned on, the scent-containing air flow K1 remaining on the scenting portion 101 side and the scent-containing air flow K2 remaining on the releasing portion 103 side pass through the space portion 102 and are sucked into the exhaust portion 104 attached to the lower portion of the space portion 102. At this time, the residual scent components F1 remaining in the space portion 102 are also sucked into the exhaust portion 104.

In a third step, the external air flow K3 taken by the intake portion 111 also passes through the space portion 102 and is sucked into the exhaust portion 104 attached to the lower portion of the space portion 102 in a state of containing the residual scent components F1 remaining in the space portion 102.

In a fourth step, the scent-containing air flow K1, the scent-containing air flow K2, and the external air flow K3, which have been sucked into the exhaust portion 104 and contain the residual scent components F1, are sent into the scent component trap portion 105 such that a scent component is trapped by the scent component trap portion 105, and are sucked into the exhaust portion 121.

In a fifth step, the scent-containing air flow K1, the scent-containing air flow K2, and the external air flow K3, which have been sucked into the exhaust portion 121, are sent to the scent component trap portion 122 such that a scent component is further trapped by the scent component trap portion 122 and deodorized, and then, are released to the outside.

According to the scent dispensing device 400 of the present embodiment, the scent component trap portion 105 further traps scent components of the air flow from which scent components has been trapped by the scent component trap portion 122, and then, releases the air flow to the outside, so that the accuracy of trapping of the scent components is improved, and a deodorizing effect can be enhanced, in addition to effects similar to those of the scent dispensing device 200 of the second embodiment.

### 5. Fifth Embodiment

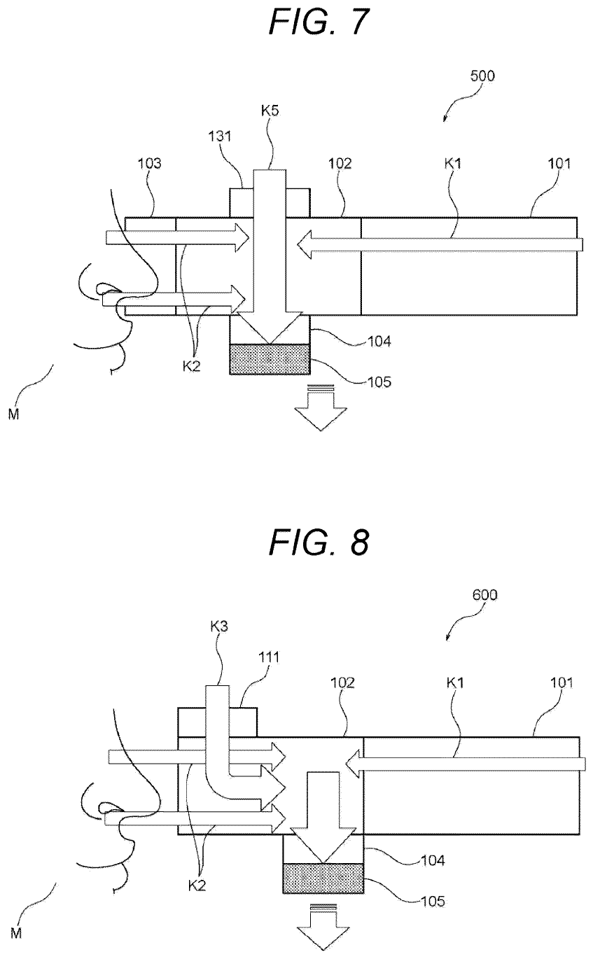

Next, a configuration example of a scent dispensing device 500 according to a fifth embodiment of the present technology will be described with reference to FIG. 7. FIG. 7 is a schematic configuration diagram illustrating the configuration example of the scent dispensing device 500. The scent dispensing device 500 differs from the scent dispensing device 200 according to the second embodiment in that an intake portion that sucks an external air flow and an exhaust portion that discharges an air flow to the outside are provided on the same straight line in the vertical direction.

As illustrated in FIG. 7, the scent dispensing device 500 includes, as an example, the scenting portion 101, the space portion 102 connected to the scenting portion 101, the releasing portion 103 connected to the space portion 102, the exhaust portion 104 attached to the space portion 102, and the scent component trap portion 105 attached to a lower portion of the exhaust portion 104 in an exhaust direction. Moreover, the scent dispensing device 500 further includes an intake portion 131 attached to the space portion 102.

The intake portion 131 is attached to, for example, an upper portion of the center of the space portion 102 in the horizontal direction, and includes an intake fan therein. Similarly, the exhaust portion 104 is attached to a lower portion of the center of the space portion 102 in the horizontal direction, and the intake portion 131 and the exhaust portion 104 are arranged on the same straight line in the vertical direction. The intake portion 111 intakes an external air flow K5 into the space portion 102 in conjunction with the exhaust portion 104.

In the operation of the deodorization process of the scent dispensing device 500, in a first step, an exhaust fan of the exhaust portion 104 and the intake fan of the intake portion 131 are turned on simultaneously when a scenting operation is ended and an air flow emission is turned off.

In a second step, when the exhaust fan and the intake fan are turned on, the scent-containing air flow K1 remaining on the scenting portion 101 side and the scent-containing air flow K2 remaining on the releasing portion 103 side pass through the space portion 102 and are sucked into the exhaust portion 104 attached to the lower portion of the space portion 102. At this time, the residual scent components F1 remaining in the space portion 102 are also sucked into the exhaust portion 104.

In a third step, the external air flow K5 taken by the intake portion 131 also passes through the space portion 102 and is sucked into the exhaust portion 104 attached to the lower portion of the space portion 102 in a state of containing the residual scent components F1 remaining in the space portion 102. At this time, since the intake portion 131 and the exhaust portion 104 are arranged on the same straight line in the vertical direction, the external air flow K5 taken by the intake portion 131 contains the residual scent components F1 and is sucked into the exhaust portion 104 without remaining in the space portion 102.

In a fourth step, the scent-containing air flow K1, the scent-containing air flow K2, and the external air flow K5, which have been sucked into the exhaust portion 104 and contain the residual scent components F1, are sent into the scent component trap portion 105 such that a scent component is trapped by the scent component trap portion 105 and deodorized, and then, are released to the outside.

According to the scent dispensing device 500 of the present embodiment, since the intake portion 131 and the exhaust portion 104 are arranged on the same straight line in the vertical direction, a deodorizing effect can be enhanced by efficiently discharging the residual scent components F1 to the exhaust portion 104 by the taken external air flow K5, in addition to effects similar to those of the scent dispensing device 200 according to the second embodiment.

### 6. Sixth Embodiment

Next, a configuration example of a scent dispensing device 600 according to a sixth embodiment of the present technology will be described with reference to FIG. 8. FIG. 8 is a schematic configuration diagram illustrating the configuration example of the scent dispensing device 600. The scent dispensing device 600 differs from the scent dispensing device 200 according to the second embodiment in terms of not including the releasing portion 103.

As illustrated in FIG. 8, the scent dispensing device 600 includes, as an example, the scenting portion 101, the space portion 102 connected to the scenting portion 101, the exhaust portion 104 attached to the space portion 102, and the scent component trap portion 105 attached to a lower portion of the exhaust portion 104 in an exhaust direction. Moreover, the scent dispensing device 200 further includes an intake portion 111 attached to the space portion 102.

The scent dispensing device 600 causes the scent-containing air flow K1 emitted from the scenting portion 101 to pass through the space portion 102 and to be emitted directly from the space portion 102 to the user M in a scenting operation of dispensing a scent to the user M. Meanwhile, an operation similar to that of the scent dispensing device 200 according to the second embodiment is performed regarding an operation of a deodorization process of the scent dispensing device 600, and thus, description thereof is omitted.

According to the scent dispensing device 600 of the present embodiment, since the releasing portion 103 that releases a scent-containing air flow to the user M is not provided, it is possible to achieve further simplification, size reduction, and weight reduction, in addition to effects similar to those of the scent dispensing device 200 of the second embodiment.

### 7. Seventh Embodiment

(1) Configuration Example of Scent Dispensing Device

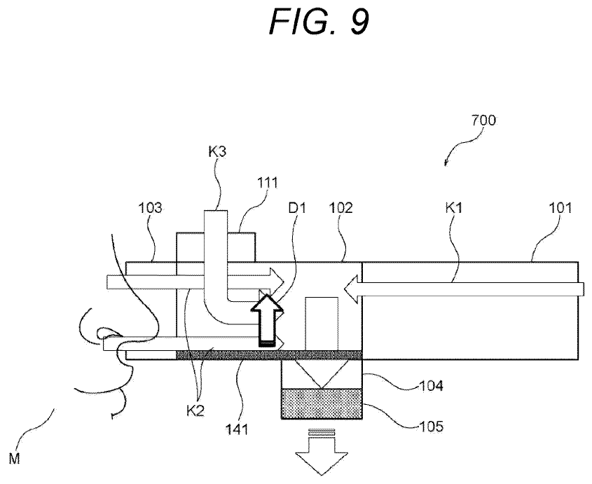

Next, a configuration example of a scent dispensing device 700 according to a seventh embodiment of the present technology will be described with reference to FIG. 9. FIG. 9 is a schematic configuration diagram illustrating the configuration example of the scent dispensing device 600. The scent dispensing device 700 differs from the scent dispensing device 200 according to the second embodiment in terms of including a deodorizing filter in the space portion 102.

As illustrated in FIG. 9, the scent dispensing device 700 includes, as an example, the scenting portion 101, the space portion 102 connected to the scenting portion 101, the releasing portion 103 connected to the space portion 102, the exhaust portion 104 attached to the space portion 102, and the scent component trap portion 105 attached to a lower portion of the exhaust portion 104 in an exhaust direction. Furthermore, the scent dispensing device 700 also includes the intake portion 111 attached to an upper portion of the space portion 102 on a side close to the releasing portion 103.

Moreover, the scent dispensing device 700 further includes a deodorizing filter 141 that adsorbs scent components remaining in the space portion 102. Note that the scent dispensing device 700 can also include a scanning portion that performs scan of the deodorizing filter 141 in the space portion 102.

The deodorizing filter 141 is arranged at a bottom in the space portion 102, and can move the space portion 102 in the vertical direction. However, an operation of the deodorizing filter 141 may be an operation in the horizontal direction, a rotating operation, or the like. Note that a power source of the deodorizing filter 141 may be an actuator or an air flow.

As the deodorizing filter 141, for example, a slit structure in which a gas adsorbing substance such as activated carbon is woven or kneaded, or a fiber or a structure body itself has a gas adsorption structure and the like can be applied. The slit structure may be a linear slit, a honeycomb structure, or the like.

(2) Configuration Example of Deodorizing Filter

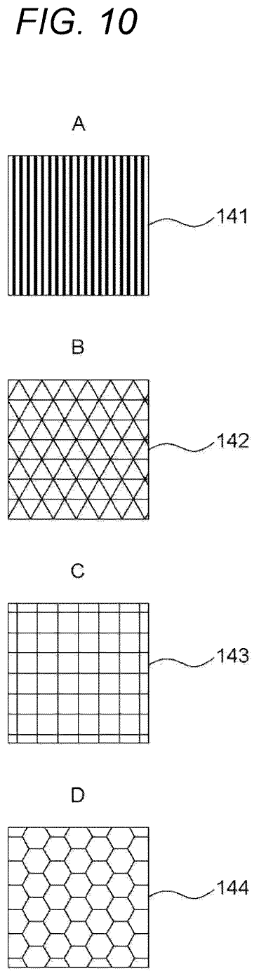

Next, configuration examples of a deodorizing filter applicable to the scent dispensing device 700 will be described with reference to FIG. 10. FIGS. 10A to 10D are schematic plan views illustrating configuration examples of the deodorizing filter applicable to the scent dispensing device 700.

As illustrated in FIG. 10A, the deodorizing filter 141 can use a slit structure having linear shapes arrayed at equal intervals. Furthermore, the deodorizing filter 142 can use a slit structure in which a plurality of triangular shapes is arrayed as illustrated in FIG. 10B. Furthermore, the deodorizing filter 143 can use a slit structure in which a plurality of rectangles is arrayed by arraying a plurality of orthogonal straight lines at equal intervals as illustrated in FIG. 100. Furthermore, the deodorizing filter 143 may have a slit structure having a honeycomb structure as illustrated in FIG. 10D.

(3) Operation Example of Scent Dispensing Device

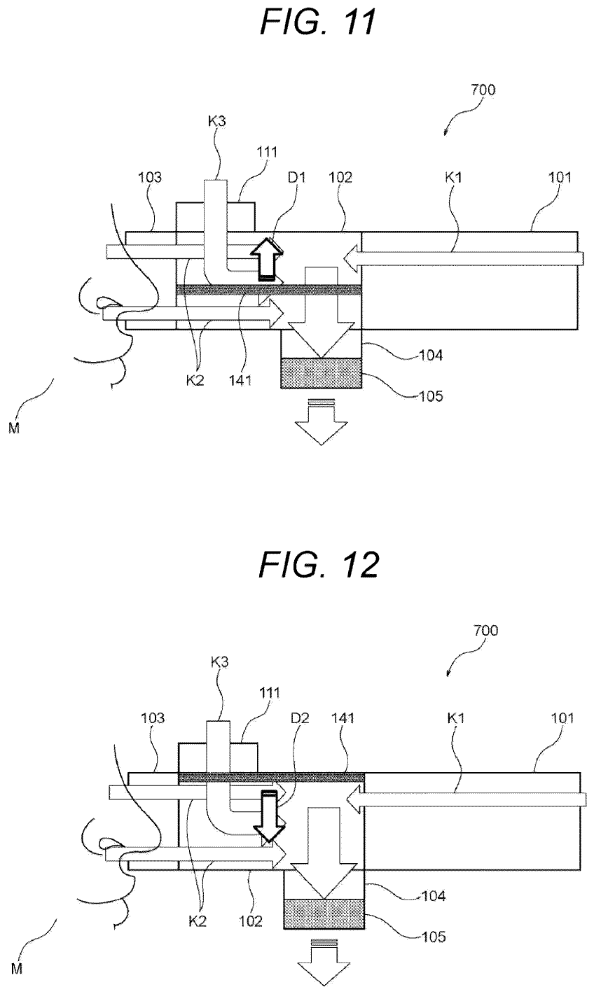

Next, operation examples of a deodorization process by the scent dispensing device 100 will be described with reference to FIGS. 9 and 11 to 13. FIGS. 11 to 13 are schematic configuration diagrams illustrating the operation examples of the scent dispensing device 700.

In the operation of the deodorization process of the scent dispensing device 700, in a first step, an exhaust fan of the exhaust portion 104 and an intake fan of the intake portion 111 are turned on simultaneously when a scenting operation is ended and an air flow emission is turned off.

In a second step, when the exhaust fan and the intake fan are turned on, the scent-containing air flow K1 remaining on the scenting portion 101 side and the scent-containing air flow K2 remaining on the releasing portion 103 side pass through the space portion 102 and are sucked into the exhaust portion 104 attached to the lower portion of the space portion 102. At this time, the residual scent components F1 remaining in the space portion 102 are also sucked into the exhaust portion 104.

In a third step, the external air flow K3 taken by the intake portion 111 also passes through the space portion 102 and is sucked into the exhaust portion 104 attached to the lower portion of the space portion 102 in a state of containing the residual scent components F1 remaining in the space portion 102.

In a fourth step, as illustrated in FIG. 9, the deodorizing filter 141 arranged at the bottom in the space portion 102 is moved upward in the movement direction D1 of the space portion 102. As illustrated in FIG. 11, the deodorizing filter 141 moves in a movement direction D1 to a ceiling in the space portion 102.

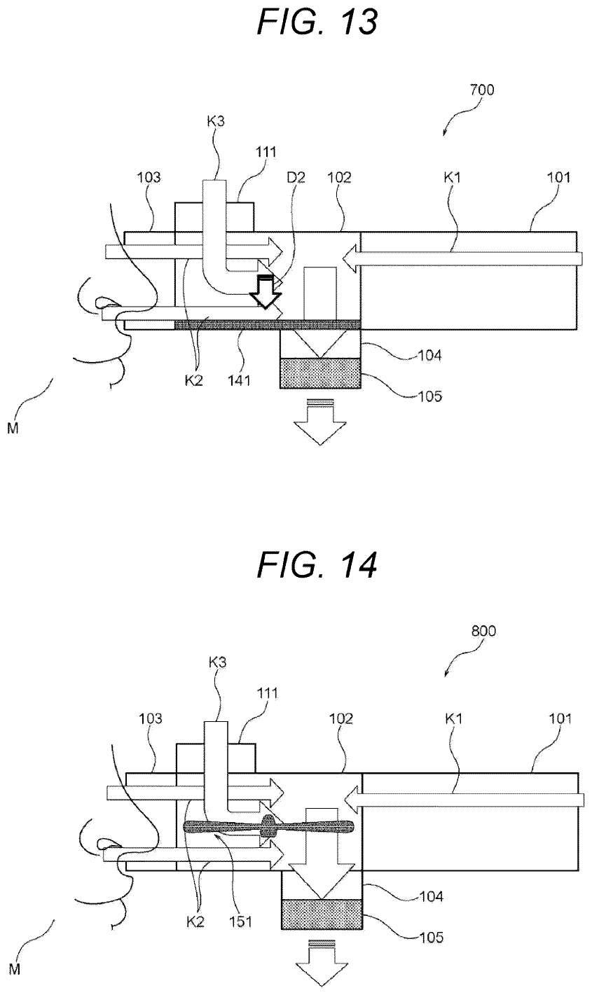

In a fifth step, as illustrated in FIG. 12, when the deodorizing filter 141 reaches the ceiling in the space portion 102, the movement direction is reversed, and the deodorizing filter 141 is moved downward in a movement direction D2 of the space portion 102. As illustrated in FIG. 13, the deodorizing filter 141 moves in the movement direction D2 to the bottom in the space portion 102. Therefore, the deodorizing filter 141 adsorbs and removes a scent component remaining in the space portion 102.

In a sixth step, the scent-containing air flow K1, the scent-containing air flow K2, and the external air flow K3, which have been sucked into the exhaust portion 104 and contain the residual scent components F1, are sent into the scent component trap portion 105 such that a scent component is trapped by the scent component trap portion 105 and deodorized, and then, are released to the outside.

According to the scent dispensing device 700 of the present embodiment, since the scent components remaining in the space portion 102 are removed by the deodorizing filter 141, a deodorizing effect in the space portion 102 can be enhanced, in addition to effects similar to those of the scent dispensing device 200 according to the second embodiment.

### 8. Eighth Embodiment

(1) Configuration Example of Scent Dispensing Device

Next, a configuration example of a scent dispensing device 800 according to an eighth embodiment of the present technology will be described with reference to FIG. 14. FIG. 14 is a schematic configuration diagram illustrating the configuration example of the scent dispensing device 800. The scent dispensing device 800 differs from the scent dispensing device 200 according to the second embodiment in terms of including a stirring mechanism in the space portion 102.

As illustrated in FIG. 14, the scent dispensing device 800 includes, as an example, the scenting portion 101, the space portion 102 connected to the scenting portion 101, the releasing portion 103 connected to the space portion 102, the exhaust portion 104 attached to the space portion 102, and the scent component trap portion 105 attached to a lower portion of the exhaust portion 104 in an exhaust direction. Furthermore, the scent dispensing device 800 also includes the intake portion 111 attached to an upper portion of the space portion 102 on a side close to the releasing portion 103.

Moreover, the scent dispensing device 800 further includes a deodorizing fan 151 which is the stirring mechanism that stirs scent components remaining in the space portion 102 for adsorption.

(2) Configuration Example of Deodorizing Fan

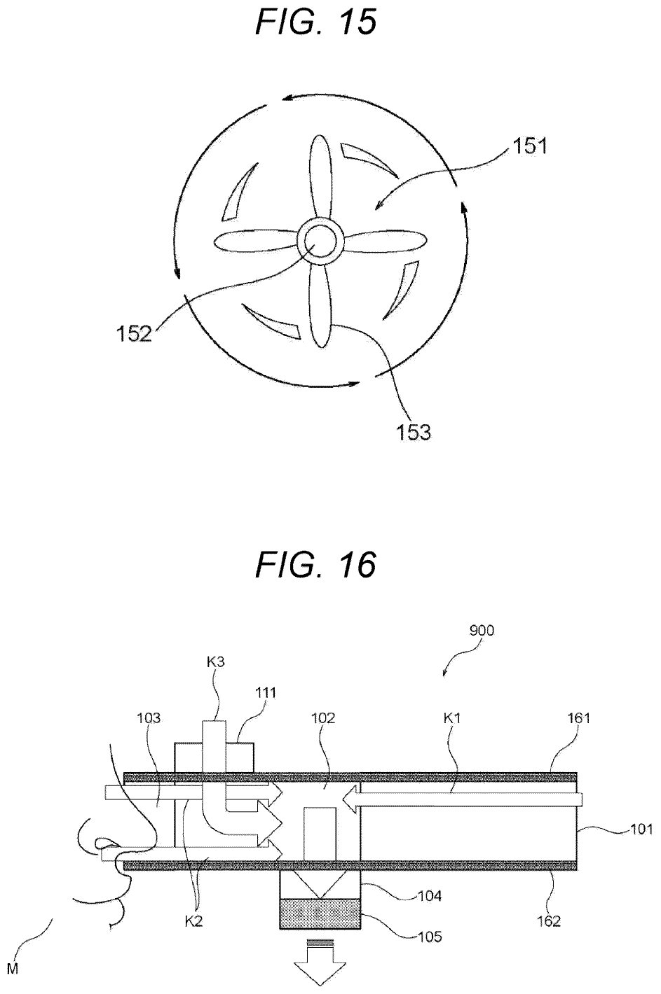

Next, a configuration example of the deodorizing fan 151 applicable to the scent dispensing device 800 will be described with reference to FIG. 15. FIG. 15 is a schematic plan view illustrating the configuration example of the deodorizing fan 151 applicable to the scent dispensing device 800.

As illustrated in FIG. 15, the deodorizing fan 151 includes a rotary shaft 152 and a plurality of rotary wings 153 attached to the rotary shaft 152. In the deodorizing fan 151, as an example, the plurality of rotary wings 153 rotates about the rotary shaft 152 counterclockwise with respect to the paper surface of FIG. 15.

(3) Operation Example of Scent Dispensing Device

Next, an operation example of a deodorization process by the scent dispensing device 800 will be described with reference to FIG. 14.

In the operation of the deodorization process of the scent dispensing device 800, in a first step, an exhaust fan of the exhaust portion 104 and an intake fan of the intake portion 111 are turned on simultaneously when a scenting operation is ended and an air flow emission is turned off.

In a second step, when the exhaust fan and the intake fan are turned on, the scent-containing air flow K1 remaining on the scenting portion 101 side and the scent-containing air flow K2 remaining on the releasing portion 103 side pass through the space portion 102 and are sucked into the exhaust portion 104 attached to the lower portion of the space portion 102. At this time, the residual scent components F1 remaining in the space portion 102 are also sucked into the exhaust portion 104.

In a third step, the external air flow K3 taken by the intake portion 111 also passes through the space portion 102 and is sucked into the exhaust portion 104 attached to the lower portion of the space portion 102 in a state of containing the residual scent components F1 remaining in the space portion 102.

In a fourth step, a rotating operation of the deodorizing fan 151 arranged in the space portion 102 is started. Therefore, a scent component remaining in the space portion 102 is stirred by the deodorizing fan 151 to be adsorbed and removed.

In a fifth step, the scent-containing air flow K1, the scent-containing air flow K2, and the external air flow K3, which have been sucked into the exhaust portion 104 and contain the residual scent components F1, are sent into the scent component trap portion 105 such that a scent component is trapped by the scent component trap portion 105 and deodorized, and then, are released to the outside.

According to the scent dispensing device 800 of the present embodiment, since the scent components remaining in the space portion 102 are removed by the deodorizing fan 151, a deodorizing effect in the space portion 102 can be enhanced, in addition to effects similar to those of the scent dispensing device 200 according to the second embodiment.

### 9. Ninth Embodiment

Next, a configuration example of a scent dispensing device 900 according to a ninth embodiment of the present technology will be described with reference to FIG. 16. FIG. 16 is a schematic configuration diagram illustrating the configuration example of the scent dispensing device 900. The scent dispensing device 900 differs from the scent dispensing device 200 according to the second embodiment in terms of including a deodorizing sheet on an inner wall surface of the device.

As illustrated in FIG. 16, the scent dispensing device 900 includes, as an example, the scenting portion 101, the space portion 102 connected to the scenting portion 101, the releasing portion 103 connected to the space portion 102, the exhaust portion 104 attached to the space portion 102, and the scent component trap portion 105 attached to a lower portion of the exhaust portion 104 in an exhaust direction. Furthermore, the scent dispensing device 900 also includes the intake portion 111 attached to an upper portion of the space portion 102 on a side close to the releasing portion 103.

Moreover, the scent dispensing device 900 includes a deodorizing sheet 161 and a deodorizing sheet 162 that cover an inner wall surface of the scenting portion 101 and/or the space portion 102 and are detachable.

The deodorizing sheet 161 covers an upper inner wall surface of the space portion 102. The deodorizing sheet 162 covers the upper inner wall surface of the space portion 102. The deodorizing sheet 161 and the deodorizing sheet 162 adsorb and remove scent components remaining in the space portion 102. Note that the deodorizing sheet 161 and the deodorizing sheet 162 can also cover upper and lower inner wall surfaces of the releasing portion 103.

In the operation of the deodorization process of the scent dispensing device 900, in a first step, an exhaust fan of the exhaust portion 104 and an intake fan of the intake portion 111 are turned on simultaneously when a scenting operation is ended and an air flow emission is turned off.

In a second step, when the exhaust fan and the intake fan are turned on, the scent-containing air flow K1 remaining on the scenting portion 101 side and the scent-containing air flow K2 remaining on the releasing portion 103 side pass through the space portion 102 and are sucked into the exhaust portion 104 attached to the lower portion of the space portion 102. At this time, the residual scent components F1 remaining in the space portion 102 are also sucked into the exhaust portion 104.

In a third step, the external air flow K3 taken by the intake portion 111 also passes through the space portion 102 and is sucked into the exhaust portion 104 attached to the lower portion of the space portion 102 in a state of containing the residual scent components F1 remaining in the space portion 102.

In a fourth step, the deodorizing sheet 161 and the deodorizing sheet 162 adsorb and remove scent components remaining in the scenting portion 101, the space portion 102, and the releasing portion 103. Note that this step can be simultaneously performed in parallel with another step.

In a fifth step, the scent-containing air flow K1, the scent-containing air flow K2, and the external air flow K3, which have been sucked into the exhaust portion 104 and contain the residual scent components F1, are sent into the scent component trap portion 105 such that a scent component is trapped by the scent component trap portion 105 and deodorized, and then, are released to the outside.

According to the scent dispensing device 900 of the present embodiment, since the scent components remaining in the space portion 102 are removed by the deodorizing sheet 161 and the deodorizing sheet 162, a deodorizing effect in the space portion 102 can be enhanced, in addition to effects similar to those of the scent dispensing device 200 according to the second embodiment.

### 10. Tenth Embodiment

Next, a configuration example of a scent dispensing device 1000 according to a tenth embodiment of the present technology will be described with reference to FIG. 17. FIG. 17 is a schematic configuration diagram illustrating the configuration example of the scent dispensing device 1000. The scent dispensing device 1000 differs from the scent dispensing device 100 according to the first embodiment in terms of having a structure in which droplets of saliva of the user M do not flow into the scent dispensing device 1000.

As illustrated in FIG. 17, the scent dispensing device 1000 includes, as an example, the scenting portion 101, the space portion 102 connected to the scenting portion 101, the releasing portion 103 connected to the space portion 102, the exhaust portion 104 attached to the space portion 102, and the scent component trap portion 105 attached to a lower portion of the exhaust portion 104 in an exhaust direction.

The scent dispensing device 1000 has a structure in which the releasing portion 103 has a nose cover, and the nose cover is mounted to a nose of the user M. Moreover, a structure body 171 having a slope shape inclined to the user M side is provided inside the nose cover. Note that the structure body 171 may be a bent portion.

Since the structure body 171 has the slope shape inclined to the user M side, even if a droplet of saliva of the user M flow into the releasing portion 103, the slope shape described above can prevent the droplet from being reflected to the user M side and flowing into the space portion 102, the scenting portion 101, and the like.

The scent dispensing device 1000 performs operations similar to those of the scent dispensing device 100 according to the first embodiment regarding a scenting operation of dispensing a scent to the user M and a deodorization process of the space portion 102, and thus, description thereof is omitted.

According to the scent dispensing device 1000 of the present embodiment, the structure body 171 can prevent the droplets from flowing into the scent dispensing device 1000, in addition to effects similar to those of the scent dispensing device 100 according to the first embodiment.

### 11. Eleventh Embodiment

(1) Configuration Example of Scent Dispensing Device

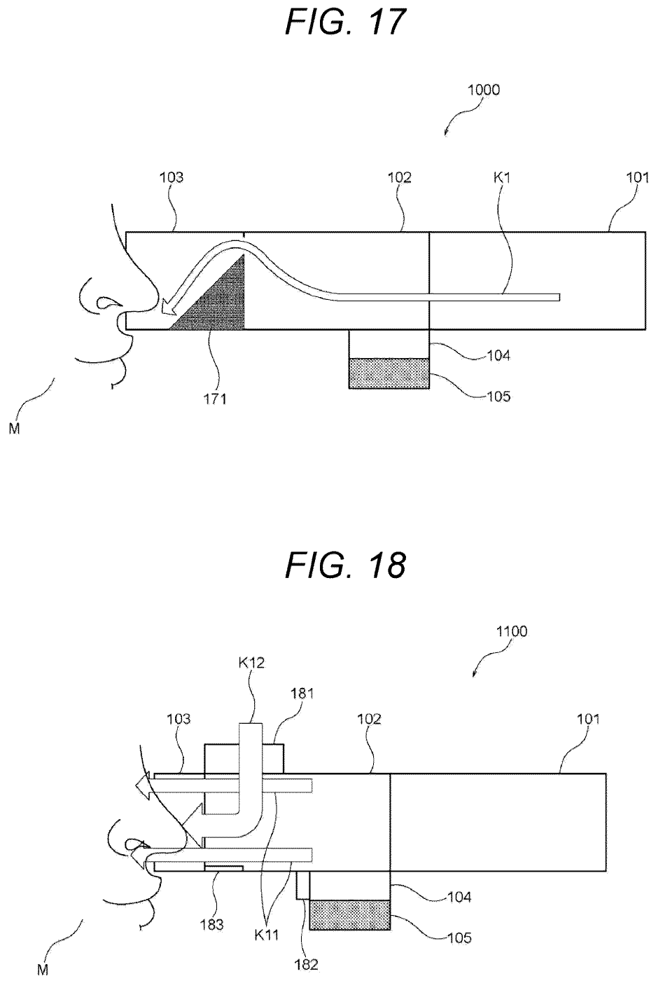

Next, a configuration example of a scent dispensing device 1100 according to an eleventh embodiment of the present technology will be described with reference to FIG. 18. FIG. 18 is a schematic configuration diagram illustrating the configuration example of the scent dispensing device 1100. The scent dispensing device 1100 differs from the scent dispensing device 200 according to the second embodiment in terms of having a function of preventing droplets of saliva of the user M from flowing into the scent dispensing device 1000.

As illustrated in FIG. 18, the scent dispensing device 1100 includes, as an example, the scenting portion 101, the space portion 102 connected to the scenting portion 101, the releasing portion 103 connected to the space portion 102, the exhaust portion 104 attached to the space portion 102, and the scent component trap portion 105 attached to a lower portion of the exhaust portion 104 in an exhaust direction. Furthermore, the scent dispensing device 1100 also includes an intake portion 181 attached to an upper portion of the space portion 102 on a side close to the releasing portion 103.

The intake portion 181 includes, for example, an intake fan therein. The intake portion 181 intakes an external air flow K12 into the space portion 102 in conjunction with the exhaust portion 104. At this time, the intake portion 181 generates an air flow by directing the external air flow K12 to a nose tip side of the user M. Therefore, even if a droplet of saliva of the user M flows into the releasing portion 103, an air shutter is generated by the external air flow K12, and thus, the droplet can be prevented from flowing into the space portion 102, the scenting portion 101, and the like.

The intake portion 181 and/or the exhaust portion 104 can include a controller 182 that controls an operation of constantly blowing the external air flow K12 taken from the intake portion 181 toward the releasing portion 103 and exhausting the air flow in the space portion 102 from the exhaust portion 104 to the outside during the exhaust.

The scent dispensing device 1100 can include a sensor 183, such as a pressure sensor, which detects an air flow flowing from the releasing portion 103 in a direction toward the space portion 102, such as a reverse flow of a scent-containing air flow directed to the user M or an inflow of the droplet. Therefore, the scent dispensing device 1100 can detect the reverse flow or the inflow air flow and operate the external air flow K12 as the air shutter, and can also stop emission of the scent-containing air flow from the scenting portion 101 when detecting the reverse flow or the inflow air flow.

(2) Arrangement Examples of Intake Portion and Exhaust Portion

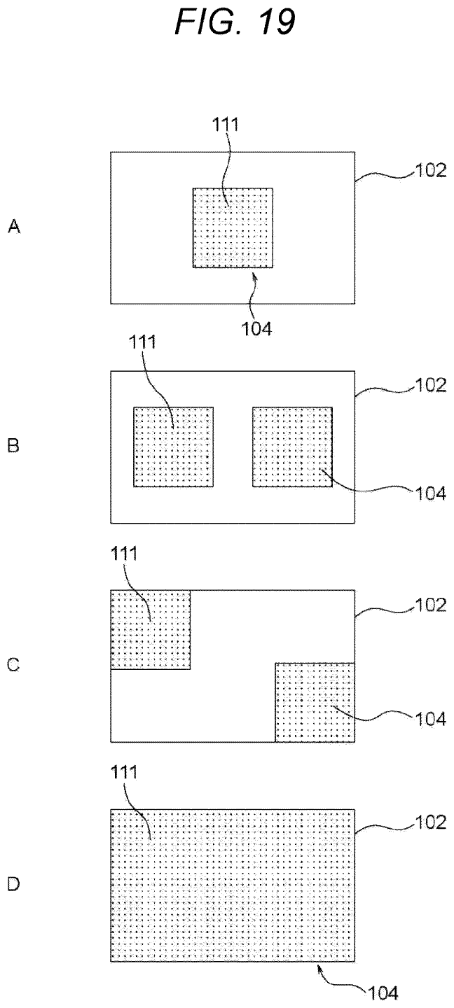

Next, arrangement examples of an intake portion and an exhaust portion applicable to the scent dispensing device 1100 will be described with reference to FIG. 19. FIGS. 19A to 19D are schematic plan views illustrating arrangement examples of the intake portion 111 and the exhaust portion 104 of the space portion 102 when viewed from above the scent dispensing device 1100. Note that any combination of the arrangement examples in FIGS. 19A to 19D can be used.

As illustrated in FIG. 19A, the intake portion 111 and the exhaust portion 104 can be arranged on the same straight line in the vertical direction in a state of completely overlapping each other in the vicinity of the center of the space portion 102 in a plan view. Furthermore, as illustrated in FIG. 19B, the intake portion 111 can be arranged on the left side of the space portion 102 in a plan view on a side close to the releasing portion 103, and the exhaust portion 104 can be arranged on the right side of the space portion 102 in a plan view on a side close to the scenting portion 101.

Furthermore, as illustrated in FIG. 19C, the intake portion 111 can be arranged at an upper left corner of the space portion 102 in a plan view, and the exhaust portion 104 can be arranged at a lower right corner of the space portion 102 in a plan view. Furthermore, as illustrated in FIG. 19D, the intake portion 111 can be arranged on the entire upper surface of the space portion 102, and the exhaust portion 104 can be arranged on the entire lower surface of the space portion 102.

(3) Operation Example of Scent Dispensing Device

Next, an operation example of a deodorization process by the scent dispensing device 1100 will be described.

The scent dispensing device 1100 performs operations similar to those of the scent dispensing device 200 according to the second embodiment as both a scenting operation of dispensing a scent to the user M and an operation of the deodorization process of the space portion 102, and thus, description thereof is omitted.

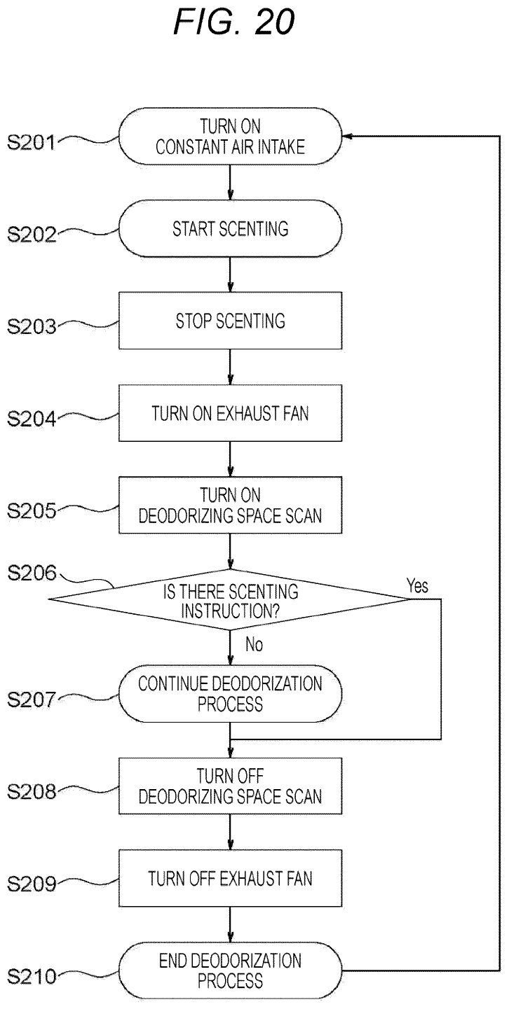

Next, an operation example of the deodorization process in a case where a plurality of the scent dispensing devices 1100 is used in conjunction with each other will be described with reference to FIG. 20. FIG. 20 is a flowchart illustrating an operation example of the plurality of scent dispensing devices 1100.

As illustrated in FIG. 20, in step S201, constant air intake is turned on at the intake portion 181 to constantly generate the external air flow K12 directed to the user M.

In step S202, a scent-containing air flow K11 formed by the scenting portion 101 is cause to pass through the space portion 102 and be released from the releasing portion 103 to start dispensing a scent to the user M (scenting).

In step S203, the air flow emission is turned off to stop the scenting operation, performed by releasing of the scent-containing air flow K11, and the dispensing of the scent to the user M is ended.

In step S204, an exhaust fan of the exhaust portion 104 is turned on in conjunction with the end of the scenting operation.

In step S205, the deodorizing filter 141 turns on deodorizing scan of the space portion 102 and performs the deodorization process in the space portion 102.

In step S206, it is determined whether or not there is a scenting instruction from the scent dispensing device 1100 different from the scent dispensing device 1100 that is performing the deodorization process. In the case of Yes, the flow proceeds to step S208. In the case of No, the flow proceeds to step S207 to continue the deodorization process.

In step S208, the deodorizing scan of the space portion 102 is turned off after the deodorization process in the space portion 102 has been sufficiently performed.

In step S209, the exhaust fan of the exhaust portion 104 is turned off.

In step S210, the deodorization process is ended. After the end of the deodorization process, the flow returns to step S201 again to start the deodorization process.

According to the scent dispensing device 1100 of the present embodiment, the external air flow K12 directed to the user M generated by the intake portion 181 can prevent droplets from flowing into the scent dispensing device 1100, in addition to effects similar to those of the scent dispensing device 200 according to the second embodiment.

The scent dispensing device according to the present technology can be utilized in, for example, an olfactory display that artificially presents a scent together with a video, and the like. Moreover, the scent dispensing device according to the present technology can also be utilized in an inspection device that measures olfactory ability, and the like.

### 12. Twelfth Embodiment

(1) Configuration Example of Scent Dispensing Device

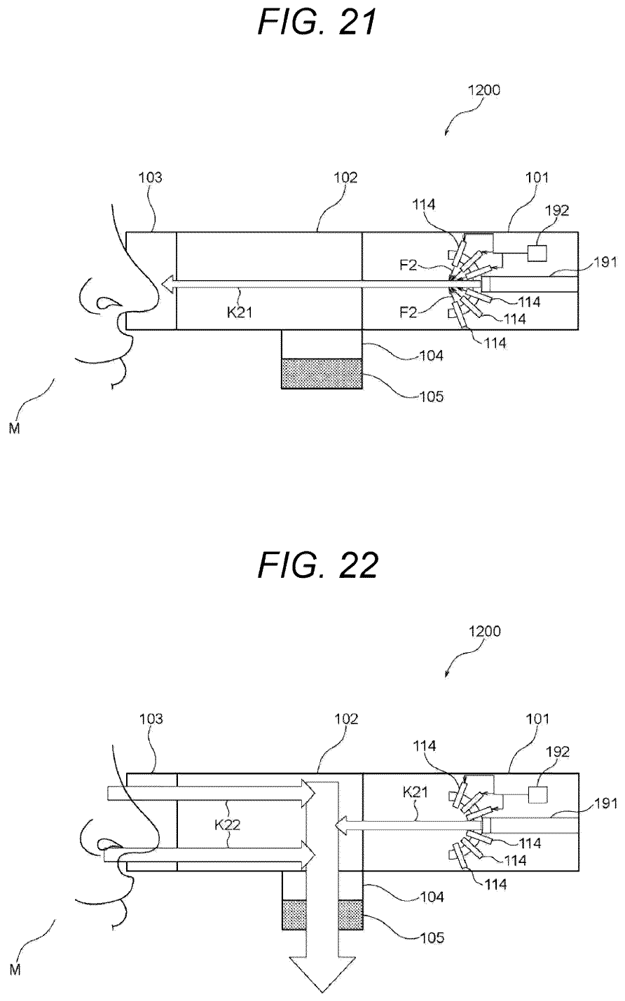

Next, a configuration example of a scent dispensing device 1200 according to a twelfth embodiment of the present technology will be described with reference to FIG. 21. FIG. 21 is a schematic configuration diagram illustrating the configuration example of the scent dispensing device 1200. The scent dispensing device 1200 differs from the scent dispensing device 100 according to the first embodiment in that the scenting portion 101 includes an intake fan sucking an external air flow, and a plurality of the perfume cartridges 114 is arranged in a fan shape.

As illustrated in FIG. 21, the scent dispensing device 1200 includes, as an example, the scenting portion 101, the space portion 102 connected to the scenting portion 101, the releasing portion 103 connected to the space portion 102, the exhaust portion 104 attached to the space portion 102, and the scent component trap portion 105 attached to a lower portion of the exhaust portion 104 in an exhaust direction.

The scenting portion 101 includes an intake fan 191 that generates a main air flow traveling straight toward a nose of the user M via the space portion 102 and the releasing portion 103. On the space portion 102 side in front of the intake fan 191, the plurality of perfume cartridges 114 is arranged in a fan shape so as to emit scent components toward the main air flow released from the intake fan 191. Moreover, the scenting portion 101 further includes a pump 192 that sends air to the plurality of perfume cartridges 114. The air containing the scent components emitted from the perfume cartridges 114 is emitted toward the main air flow and sent to the nose of the user M in a state of being carried on the main air flow. Note that the main air flow generated from the intake fan 191 does not need to travel straight, and the plurality of perfume cartridges 114 may be arranged in a spherical shape or the like.

(2) Operation Example of Scent Dispensing Device

Next, an operation example of the scent dispensing device 1200 will be described with reference to FIGS. 21 and 22. FIG. 22 is a schematic configuration diagram illustrating the operation example of the scent dispensing device 1200.

In a scenting operation of the scent dispensing device 1200, in a first step, a perfume retainer is made to retain a perfume of a scent to be dispensed to the user M, and the plurality of perfume cartridges 114 each having the perfume retainer is arranged in a fan shape and incorporated into the scenting portion 101.

In the second step, emission of the main air flow generated by the intake fan 191 is constantly turned on to cause the air flow to flow into the scenting portion 101. Then, air blowing of the pump 192 is turned on to send air to the plurality of perfume cartridges 114.

In a third step, scent components F2 are emitted from the plurality of perfume cartridges 114 toward the main air flow. When the main air flow that has flowed into the scenting portion 101 comes into contact with the scent components F2, thereby forming a scent-containing air flow K21 containing the scent. The formed scent-containing air flow K21 is sent to the nose of the user M.

In an operation of a deodorization process of the scent dispensing device 1200, in a first step, the scenting operation is ended, intake air supply of the pump 192 is turned off, and an exhaust fan of the exhaust portion 104 is turned on. Note that the scent dispensing device 1200 can also perform an operation of increasing an intake air volume by increasing a rotational speed of the intake fan 191 as the exhaust fan of the exhaust portion 104 is turned on.

In a second step, when the exhaust fan is turned on, the scent-containing air flow K21 remaining on the scenting portion 101 side and a scent-containing air flow K22 remaining on the releasing portion 103 side pass through the space portion 102 and are sucked into the exhaust portion 104 attached to the lower portion of the space portion 102. At this time, the residual scent components F1 remaining in the space portion 102 are also sucked into the exhaust portion 104.

In a third step, the scent-containing air flow K21 and the scent-containing air flow K22, which have been sucked into the exhaust portion 104 and contain the residual scent components F1, are sent into the scent component trap portion 105 such that a scent component is trapped by the scent component trap portion 105 and deodorized, and then, are released to the outside.

According to the scent dispensing device 1200 of the present embodiment, since the air containing the scent component is sent to the nose of the user M in the state of being carried on the main air flow generated from the intake fan 191 during the scenting operation, it is possible to reduce retention of a residual scent in the scenting portion 101, the space portion 102, and the releasing portion 103, and adhesion of a scent component to inner wall surfaces of these structures, in addition to effects similar to those of the scent dispensing device 100 of the first embodiment.

Note that the present technology can have the following configurations.

(1)

A scent dispensing device including:
a scenting portion that emits a scent-containing air flow containing a scent;
a space portion that causes the scent-containing air flow emitted from the scenting portion to pass or remain;
a releasing portion that releases the scent-containing air flow having passed through the space portion to a user;
an exhaust portion that exhausts the scent-containing air flow remaining in the space portion to the outside; and
a scent component trap portion that traps a volatile component in the scent-containing air flow exhausted by the exhaust portion.

(2)

The scent dispensing device according to (1), in which the exhaust portion performs an exhausting operation in conjunction with an operation of releasing the scent-containing air flow.

(3)

The scent dispensing device according to (1) or (2), in which the space portion includes a guide portion that guides the scent-containing air flow toward the scenting portion and/or a nose tip of the user.

(4)

The scent dispensing device according to any one of (1) to (3), in which the scenting portion includes a plurality of perfume retaining members each retaining a perfume.

(5)

The scent dispensing device according to any one of (1) to (4), in which the releasing portion includes a nose cover that is detachable.

(6)

The scent dispensing device according to (5), in which the nose cover is formed in a structure to be mounted to a nose of the user, and has a slope or a bent portion inclined to a side of the user inside the nose cover.

(7)

The scent dispensing device according to any one of (1) to (6), further including an intake portion that takes an external air flow into the space portion in conjunction with the exhaust portion.

(8)

The scent dispensing device according to (7), in which the intake portion includes a rectifying mechanism that blows the taken external air flow toward an outlet through which the scent-containing air flow of the scenting portion is emitted.

(9)

The scent dispensing device according to any one of (1) to (8), further including: a deodorizing filter that adsorbs the volatile component remaining in the space portion; and a scanning portion that performs scan of the deodorizing filter in the space portion.

(10)

The scent dispensing device according to any one of (1) to (9), further including a stirring mechanism that stirs the volatile component remaining in the space portion to be adsorbed.

(11)

The scent dispensing device according to any one of (1) to (10), further including a deodorizing sheet that covers an inner wall surface of the scenting portion and/or the space portion and is detachable.

(12)

The scent dispensing device according to any one of (7) to (11), further including a controller that controls an operation of constantly blowing the external air flow, taken from the intake portion, to the releasing portion and exhausting an air flow in the space portion from the exhaust portion to the outside during exhaust.

(13)

The scent dispensing device according to any one of (1) to (12), further including a sensor that detects an air flow flowing from the releasing portion in a direction toward the space portion, in which the scenting portion stops emitting the scent-containing air flow when the flowing air flow is detected.

(14)

A scent removing method including:
a step of emitting a scent-containing air flow containing a scent;
a step of causing the emitted scent-containing air flow to pass through an enclosed space;
a step of exhausting the scent-containing air flow remaining in the enclosed space to the outside; and
a step of trapping a volatile component in the scent-containing air flow to be exhausted.

### REFERENCE SIGNS LIST

100, 200, 300, 400, 500, 600, 700, 800, 900, 1000, 1100, 1200 Scent dispensing device
101 Scenting portion
102 Space portion
103 Releasing portion
104, 121 Exhaust portion
105, 122 Scent component trap portion
111, 112, 131, 181 Intake portion
113 Flap
114 Perfume cartridge
141 to 144 Deodorizing filter
151 Deodorizing fan (stirring mechanism)
152 Rotary shaft
153 Rotary wing
161, 162 Deodorizing sheet
171 Structure body
182 Controller
183 Sensor
191 Intake fan
192 Pump
M User
K1, K2, K11, K21, K22 Scent-containing air flow
K3, K4, K5, K12 External air flow
F1 Residual scent component
F2 Scent component

1. A scent dispensing device comprising:
a scenting portion that emits a scent-containing air flow containing a scent;
a space portion that causes the scent-containing air flow emitted from the scenting portion to pass, remain, or be mixed;
a releasing portion that releases the scent-containing air flow having passed through the space portion to a user;
an exhaust portion that exhausts the scent-containing air flow remaining in the space portion to an outside; and
a scent component trap portion that traps a volatile component in the scent-containing air flow exhausted by the exhaust portion.

2. The scent dispensing device according to claim 1, wherein the exhaust portion performs an exhausting operation in conjunction with an operation of releasing the scent-containing air flow.

3. The scent dispensing device according to claim 1, wherein the space portion includes a guide portion that guides the scent-containing air flow toward the scenting portion and/or a nose tip of the user.

4. The scent dispensing device according to claim 1, wherein the scenting portion includes a plurality of perfume retaining members each retaining a perfume.

5. The scent dispensing device according to claim 1, wherein the releasing portion includes a nose cover that is detachable.

6. The scent dispensing device according to claim 5, wherein the nose cover is formed in a structure to be mounted to a nose of the user, and has a slope or a bent portion inclined to a side of the user inside the nose cover.

7. The scent dispensing device according to claim 1, further comprising an intake portion that takes an external air flow into the space portion in conjunction with the exhaust portion.

8. The scent dispensing device according to claim 7, wherein the intake portion includes a rectifying mechanism that blows the taken external air flow toward an outlet through which the scent-containing air flow of the scenting portion is emitted.

9. The scent dispensing device according to claim 1, further comprising: a deodorizing filter that adsorbs the volatile component remaining in the space portion; and a scanning portion that performs scan of the deodorizing filter in the space portion.

10. The scent dispensing device according to claim 1, further comprising a stirring mechanism that stirs the volatile component remaining in the space portion to be adsorbed.

11. The scent dispensing device according to claim 1, further comprising a deodorizing sheet that covers an inner wall surface of the scenting portion and/or the space portion and is detachable.

12. The scent dispensing device according to claim 7, further comprising a controller that controls an operation of constantly blowing the external air flow, taken from the intake portion, to the releasing portion and exhausting an air flow in the space portion from the exhaust portion to the outside during exhaust.

13. The scent dispensing device according to claim 1, further comprising a sensor that detects an air flow flowing from the releasing portion in a direction toward the space portion, wherein the scenting portion stops emitting the scent-containing air flow when the flowing air flow is detected.

14. A scent removing method comprising:
a step of emitting a scent-containing air flow containing a scent;
a step of causing the emitted scent-containing air flow to pass through an enclosed space;
a step of exhausting the scent-containing air flow remaining in the enclosed space to an outside; and
a step of trapping a volatile component in the scent-containing air flow to be exhausted.
